(19) United States
(12) Patent Application Publication
(10) Pub. No.: US 2009/0292012 A1
(43) Pub. Date: Nov. 26, 2009

(54) Therapeutic Agent

(76) Inventors: Tatsuji Enoki, Shiga (JP); Yoko Kudo, Shiga (JP); Katsumi Sugiyama, Shiga (JP); Hiromu Ohnogi, Shiga (JP); Hiroaki Sagawa, Shiga (JP); Ikunoshin Kato, Shiga (JP)

Correspondence Address: BIRCH STEWART KOLASCH & BIRCH, PO BOX 747, FALLS CHURCH, VA, 22040-0747, US

(21) Appl. No.: 11/795,806

(22) PCT Filed: Jan. 26, 2006
(86) PCT No.: PCT/JP06/01200

§ 371 Date: Jul. 23, 2007

(30) Foreign Application Priority Data
Feb. 4, 2005 (JP) 2005-029596

(51) Int. Cl. A61K 31/366; A61K 36/23; C07D 493/04

(52) U.S. Cl. 514/455; 424/725; 549/282

(57) ABSTRACT

A therapeutic agent or prophylactic agent for a disease requiring an anti-foaming action for a cell and/or an inhibitory action for acyl-CoA cholesterol acyltransferase in treatment or prevention, characterized in that the therapeutic agent or prophylactic agent comprises as an effective ingredient a processed product derived from Peucedanum japonicum.

### TECHNICAL FIELD

The present invention relates to a medicament, food or feed useful for treating or preventing a disease requiring an anti-foaming action for a cell and/or an inhibitory action for acyl-CoA cholesterol acyltransferase in treatment or prevention, for example, arteriosclerosis, hyperlipemia, or the like.

### BACKGROUND ART

In recent years, with a change of diet richly containing high-calorie food and high-cholesterol food, or an increase of old generations by rise of average longevity, so-called lifestyle-related diseases such as arteriosclerosis, hyperlipemia, hypertension, and diabetes have rapidly increased, and have become a big social problem.

In an early lesion of arteriosclerosis, a patchy or linear fat deposition called a fatty streak is observed. This change is mainly caused by an accumulation of a foamed macrophage in a vascular endothelium. A macrophage introduces a modified LDL to produce free cholesterol, and the free cholesterol is esterified by acyl-CoA cholesterol acyltransferase and the esterified cholesterol is accumulated, whereby foaming of the macrophage is caused. The above-mentioned early foamed cellular lesion progresses to a complex lesion including foaming of a vascular smooth muscle cell. The fatty streak becomes a fibrous plaque, and projects to the vessel wall with the passage of time. When the lesion further progresses, along with the calcification and adhesion of thrombus, the vascular cavity is narrowed, and the plaque is broken to cause thrombotic occlusion. In addition, it is known that a plaque which is likely to break richly contains a lipid component such as cholesterol ester. Therefore, foaming of macrophage, vascular smooth muscle cell, or the like is suppressed, or an activity of the acyl-CoA cholesterol acyltransferase is inhibited, whereby stabilization and involution of a lesion of arteriosclerosis is provided, so that there can be expected to lead to a lowering of crisis or recurrence of acute coronary syndromes based on arteriosclerosis or hyperlipemia. In addition, it is known that an activity of the acyl-CoA cholesterol acyltransferase is inhibited, whereby synthesis of the cholesterol ester constituting VLDL is lowered to decrease the VLDL synthesis in the liver or an ester bond of cholesterol is inhibited to lower the absorption of the cholesterol in the small intestine. Therefore, there can be expected to decrease a cholesterol in the blood and a triglyceride in the blood.

Botan-bofu, which has a nomenclature of Peucedanum japonicum, is a perennial herb belonging to Umbelliferae Peucedanum L. and grows in coastal scrag or grass. Peucedanum japonicum has the stem of from 30 to 100 cm in height, and produces many small white flowers in flower season from June to September. Regarding physiological activity of Peucedanum japonicum, it is said to have effects on cold, recovery from fatigue, and nutritional fortification in Okinawa where is a place of origin, and has been appreciated since early times. In recent studies, it is known that Peucedanum japonicum have a suppressive action for cancer (for example, Non-Patent Publication 1), an inhibitory action for an enzyme catabolizing disaccharide (for example, Patent Publication 1), an antioxidant action (for example, Patent Publication 2 and Non-Patent Publication 2), a cell activating action (for example, Patent Publication 2), and a suppressive action for producing melanine (for example, Patent Publication 2).

As characteristic chemical component contained in Peucedanum japonicum, multiple sorts of coumarin derivatives have been known, and a suppressive action for tumor promoter of the coumarin derivatives and the like have been studied (for example, Non-Patent Publication 3).

### Patent Publication 1: Japanese Patent Laid-Open No. 2003-26694

### Patent Publication 2: Japanese Patent Laid-Open No. 2004-26697

Non-Patent Publication 1: T. Morioka and eight others, Cancer Letters, 2004, Vol. 205, p 133-141
Non-Patent Publication 2: M. Hisamoto and two others, J. Agric. Food Chem., 2004, Vol. 52, p 445-450
Non-Patent Publication 3: B. Fan and five others, Journal of Japanese Botany, 2000, Vol. 75, No. 4, p 257-261

### DISCLOSURE OF INVENTION

### Problems to be Solved by the Invention

An object of the present invention is to develop a substance having an anti-foaming action for a cell suitable as food materials and medicament materials, which is safe and conveniently ingestible, and provide a medicament, food or feed using the composition or the substance.

### Means to Solve the Problems

Summarizing the present invention, a first invention of the present invention relates to a therapeutic agent or prophylactic agent for a disease requiring an anti-foaming action for a cell and/or an inhibitory action for acyl-CoA cholesterol acyltransferase in treatment or prevention, characterized in that the therapeutic agent or prophylactic agent comprises as an effective ingredient a processed product derived from Peucedanum japonicum.

A second invention of the present invention relates to an anti-foaming agent for a cell, characterized in that the anti-foaming agent comprises as an effective ingredient a processed product derived from Peucedanum japonicum.

A third invention of the present invention relates to an inhibitory agent for acyl-CoA cholesterol acyltransferase, characterized in that the inhibitory agent comprises as an effective ingredient a processed product derived from Peucedanum japonicum.

A fourth invention of the present invention relates to a food or feed for anti-foaming for a cell and/or an inhibition for acyl-CoA cholesterol acyltransferase, characterized in that the food or feed comprises as an effective ingredient a processed product derived from Peucedanum japonicum.

A fifth invention of the present invention relates to a therapeutic agent or prophylactic agent for a disease requiring an anti-foaming action for a cell and/or an inhibitory action for acyl-CoA cholesterol acyltransferase in treatment or prevention, characterized in that the therapeutic agent or prophylactic agent comprises as an effective ingredient one or more kinds selected from the group consisting of 3-acetoxy-4′-senecioyloxy-3′,4′-dihydroseselin, a derivative thereof, and pharmaceutically acceptable salts thereof.

A sixth invention of the present invention relates to an anti-foaming agent for a cell, characterized in that the anti-foaming agent comprises as an effective ingredient one or more kinds selected from the group consisting of 3′-acetoxy-4′-senecioyloxy-3′,4′-dihydroseselin, a derivative thereof, and pharmaceutically acceptable salts thereof.

A seventh invention of the present invention relates to an inhibitory agent for acyl-CoA cholesterol acyltransferase, characterized in that the inhibitory agent comprises as an effective ingredient one or more kinds selected from the group consisting of 3′-acetoxy-4′-senecioyloxy-3′,4′-dihydroseselin, a derivative thereof, and pharmaceutically acceptable salts thereof.

A eighth invention of the present invention relates to a food or feed for anti-foaming for a cell and/or an inhibition for acyl-CoA cholesterol acyltransferase, characterized in that the food or feed comprises as an effective ingredient one or more kinds selected from the group consisting of 3′-acetoxy-4′-senecioyloxy-3′,4′-dihydroseselin, a derivative thereof, and pharmaceutically acceptable salts thereof.

A ninth invention of the present invention relates to a method of treating or preventing a disease requiring an anti-foaming action for a cell and/or an inhibitory action for acyl-CoA cholesterol acyltransferase in treatment or prevention, comprising administering to a subject an effective amount of a processed product derived from Peucedanum japonicum.

A tenth invention of the present invention relates to use of a processed product derived from Peucedanum japonicum for manufacturing of a therapeutic agent or prophylactic agent for a disease requiring an anti-foaming action for a cell and/or an inhibitory action for acyl-CoA cholesterol acyltransferase in treatment or prevention.

A eleventh invention of the present invention relates to a method of treating or preventing a disease requiring an anti-foaming action for a cell and/or an inhibitory action for acyl-CoA cholesterol acyltransferase in treatment or prevention, comprising administering to a subject an effective amount of one or more kinds selected from the group consisting of 3′-acetoxy-4′-senecioyloxy-3′,4′-dihydroseselin, a derivative thereof, and pharmaceutically acceptable salts thereof.

A twelfth invention of the present invention relates to use of one or more kinds selected from the group consisting of 3′-acetoxy-4′-senecioyloxy-3′,4′-dihydroseselin, a derivative thereof, and pharmaceutically acceptable salts thereof for manufacturing of a therapeutic agent or prophylactic agent for a disease requiring an anti-foaming action for a cell and/or an inhibitory action for acyl-CoA cholesterol acyltransferase in treatment or prevention.

### EFFECTS OF THE INVENTION

According to the present invention, there is provided a medicament, food or feed for treating or preventing a disease requiring an anti-foaming action for a cell and/or an inhibitory action for acyl-CoA cholesterol acyltransferase in treatment or prevention. The medicament is useful for arteriosclerosis, hyperlipemia, or a disease caused by these diseases. Also, by taking the food as foodstuff on a daily basis, symptoms of a disease requiring an anti-foaming action for a cell and/or an inhibitory action for acyl-CoA cholesterol acyltransferase in treatment or prevention can be ameliorated and the like. Therefore, the food of the present invention is a functional food useful in maintaining homeostasis of a living body according to an anti-foaming action for a cell and/or an inhibitory action for acyl-CoA cholesterol acyltransferase.

### BEST MODE FOR CARRYING OUT THE INVENTION

Foaming of a cell is caused by accumulation of a cholesterol ester within the cell. An anti-foaming action for a cell can be conveniently determined by employing an assay system using the amount of the cholesterol ester in the cell as an index as described in the Example 1 set forth below. More specifically, macrophage is cultured in the presence of a test substance and acetyl LDL to evaluate the amount of the cholesterol ester in the cell, whereby an anti-foaming action for a cell can be conveniently determined. A target cell for anti-foaming in the present invention is not particularly limited, and exemplified by, for example, a vascular cell such as macrophage or a smooth muscle cell, and a blood cell.

Acyl-CoA cholesterol acyltransferase (also referred as acyl-CoA: cholesterol O-acyltransferase, hereinafter abbreviated as ACAT in some cases) is an enzyme which transfers a long-chain fatty acid from acyl-CoA to cholesterol and catalyzes the cholesterol ester synthesis. An inhibitory action for ACAT can be conveniently determined by employing an assay system as described in the Example 2 set forth below. More specifically, a test substance and an enzyme source containing ACAT are mixed to evaluate a transfer of an oleoyl group from oleoyl-CoA labeled by radiation to cholesterol, and whereby an inhibitory action for ACAT can be conveniently determined.

Peucedanum japonicum usable in the present invention is not particularly limited, and fruit, seed, seed coat, flower, leaf, stem, root, rhizome and/or whole plant can be used as it is.

The processed product derived from Peucedanum japonicum usable as an effective ingredient of the present invention (hereinafter referred as the processed product of the present invention in some cases) is not particularly limited, as long as the processed product is obtained by subjecting a raw material plant to some sort of processing and has an anti-foaming action for a cell and/or an inhibitory action for ACAT. The processed product refers to, for example, an extract, a powder, a squeezed juice, a pulverized product, a chemically processed product, or an enzymatically processed product, and is especially preferably exemplified by an extract, a powder and a squeezed juice. As the processed product of the present invention, a processed product containing 3′-acetoxy-4′-senecioyloxy-3′,4′-dihydroseselin set forth below, which is a substance having an anti-foaming action for a cell and a substance having an inhibitory action for ACAT, in a high content can be particularly preferably used. The expression “contained . . . in a high content” as used herein means that the concentration of 3′-acetoxy-4′-senecioyloxy-3′,4′-dihydroseselin in the processed product, is higher than the concentration of 3′-acetoxy-4′-senecioyloxy-3′,4′-dihydroseselin in the raw material plant. The concentration of 3′-acetoxy-4′-senecioyloxy-3′,4′-dihydroseselin in the processed product is preferably 1.5 times or more, and more preferably 2 times or more as the concentration in Peucedanum japonicum.

In the present invention, the extract refers to a substance itself obtained through the process of subjecting a raw material plant to an extraction procedure with an extraction solvent. The extraction can be carried out as follows by a known extraction method. For example, the raw material is powdered or cut into thin pieces, and thereafter extracted in a batch process or continuous process using a solvent. The extraction solvent used upon obtaining an extract is not particularly limited, and includes, for example, water, glycerol, glycols (ethylene glycol, propylene glycol, and the like), alcohols (ethanol, methanol, isopropyl alcohol, and the like), ketones (acetone, methyl ethyl ketone, and the like), and hydrophilic or lipophilic solvents (chloroform, methyl acetate, ethyl acetate, and the like), which can be used alone or properly as a mixed solution as desired. Example of using a mixed solution as the extraction solvent is not particularly limited, for example, various types of aqueous solutions can be used, and for example, a 10 to 95%, preferably a 15 to 90%, and further preferably a 20 to 85% alcohol aqueous solution can be used. In addition, also in a case where glycerol or glycols (ethylene glycol, propylene glycol, and the like) is used as a solvent, it is preferable that the solvent is used as an aqueous solution thereof. The amount of the extraction solvent may be appropriately determined. Usually, the extraction solvent may be used in an amount of the weight of the raw material plant in the form as it is upon use (for example, if the raw material plant is a raw plant, the weight of the raw plant), preferably in an amount of from 0.1- to 100-folds by weight of the raw materials. The extraction temperature may be also appropriately determined according to its purposes. In the case of the water extraction, usually, the extraction temperature is in the range of preferably from 4° to 130° C., more preferably from 25° to 100° C. Alternatively, in the case where ethanol is contained in the solvent, the extraction temperature is preferably within the range of from 4° to 60° C., from the viewpoint of safety. The extraction time may be also determined in consideration of extraction efficiency. It is usually preferable that the raw materials, the extraction solvent and the extraction temperature are set so that the extraction time is within the range of preferably from several seconds to several days, more preferably 5 minutes to 24 hours. The extraction procedure may be carried out, for example, while stirring or allowing the mixture to stand. Also, the extraction procedures may be repeated several times as desired. By the above procedures, an extract derived from Peucedanum japonicum usable in the present invention (hereinafter referred to as the extract of the present invention in some cases) can be obtained. The extract is subjected to such a process as filtration, centrifugation, concentration, ultrafiltration or molecular sieving as desired, whereby an extract in which a substance having an anti-foaming action for a cell or a substance having an inhibitory action for ACAT, for example, 3′-acetoxy-4′-senecioyloxy-3′,4′-dihydroseselin set forth below is concentrated, can be prepared. An anti-foaming action for a cell or an inhibitory action for ACAT of the extract or concentrated extract can be conveniently determined in accordance with the method described in Example 1 and 2 set forth below. Alternatively, Peucedanum japonicum usable in the present invention may be processed in the form like tea-leaves by a known method, and an extract obtained from the tea-leaf-like products can be used as the extract of the present invention as long as the extract has an anti-foaming action for a cell and/or an inhibitory action for ACAT. In addition, two or more kinds of the above extracts can be contained to be used. Also, two or more kinds of extracts obtained by different extraction methods from the raw material plants can be contained to be used.

In addition, in the present invention, a fraction obtained by fractionating the extract of the present invention by a known method, or a fraction obtained by repeating the fractionation procedures a plural times is also encompassed in the extract of the present invention. The above-mentioned fractionation means include extraction, separation by precipitation, column chromatography, thin-layer chromatography, and the like. The substance having an anti-foaming action for a cell or the substance having an inhibitory action for ACAT can also be isolated by further proceeding the purification of the resulting fraction using the anti-foaming action for a cell or the inhibitory action for ACAT as an index.

Alternatively, a processed product derived from Peucedanum japonicum usable in the present invention other than the extract of the present invention as mentioned above is exemplified by, for example, a powder derived from Peucedanum japonicum usable in the present invention (which may be hereinafter referred to as the powder of the present invention in some cases). As a method for preparing a powder of the present invention, for example, a plant is dried and powdered with a powdering machine, whereby a powder derived from Peucedanum japonicum can be obtained. In addition, the powder may be obtained by lyophilization.

In addition, the squeezed juice derived from Peucedanum japonicum usable in the present invention can be used as a processed product of the present invention. The method for preparing the squeezed juice is not particularly limited, as long as the method is a known method of squeezing a plant. For example, the method includes a method using a squeezer of a screw-type, a gear-type, a cutter-type or the like, or a juicer. Also, the raw materials may be cut into thin pieces or mashed as a pre-processing, and thereafter squeezed with the above-mentioned juicer or cloth or the like, whereby a squeezed juice can be obtained.

A pulverized product refers to one prepared by pulverizing the raw material plant, and its tissue piece is generally larger than the powder. For example, the pulverized product can be prepared by using a pulverizer. Also, the chemically processed product is not particularly limited, and refers to a product obtained by subjecting the raw material plant to an acid processing, an alkali processing, an oxidation processing, a reducing processing or the like. The chemically processed product can be prepared, for example, by immersing the raw material plant in an aqueous solution containing an inorganic acid or organic acid, such as hydrochloric acid, sulfuric acid, nitric acid, citric acid or acetic acid, or an inorganic base or organic base, such as sodium hydroxide, potassium hydroxide or ammonia. The chemically processed product includes all those derived from plants subjected to chemical processing as mentioned above. The enzymatically processed product refers, for example, to an enzymatically processed product processed with pectinase, cellulase, xylanase, amylase, mannanase, or glucosidase, an enzymatic reaction product by a microorganism (for example, a fermented product) or the like. The enzymatically processed product can be prepared, for example, by allowing the above-mentioned enzyme to act on the raw material plant in an appropriate buffer. The enzymatically processed product includes all those derived from plants subjected to the enzymatic processing as mentioned above. Further, the processed product derived from Peucedanum japonicum of the present invention encompasses, for example, juice obtained by cutting the stem of the raw material plant and obtaining juice from its cross section.

In the present invention, the shape of the processed product of the present invention is not particularly limited as long as the processed product can exhibit an anti-foaming action for a cell and/or an inhibitory action for ACAT when the processed product is used as an effective ingredient in each of the embodiments of the present invention, and the processed product may take any form of powder, solid or liquid. In addition, the processed product can be used in the form of a granular solid prepared by granulating the processed product by a known process. The granulation process is not particularly limited, and is exemplified by tumbling granulation, agitation granulation, fluidizing bed granulation, airflow granulation, extruding granulation, compression molding granulation, disintegration granulation, spray granulation, spray-dry granulation or the like. In addition, the powdery processed product can be used as the processed product derived from Peucedanum japonicum of the present invention in the form of a liquid prepared by dissolving the powdery processed product in a liquid, for example, water, an alcohol or the like.

In the present invention, as a processed product containing 3′-acetoxy-4′-senecioyloxy-3′,4′-dihydroseselin set forth below in a high content preferably usable as a processed product of the present invention, for example, an ethanol extract, a water-containing ethanol extract, a hydrothermal extract, a glycerol extract or a water-containing glycerol extract, a glycol extract or a water-containing glycol extract such as ethylene glycol or propylene glycol, or a powder thereof is preferable from the viewpoint of containing the compound and further being used for edible purposes.

In addition, the present inventors have searched a substance having an anti-foaming action for a cell and a substance having an inhibitory action for ACAT derived from Peucedanum japonicum and have found that 3-acetoxy-4′-senecioyloxy-3′,4′-dihydroseselin represented by the following formula (I) is a substance having an anti-foaming action for a cell and a substance having an inhibitory action for ACAT. More specifically, as an effective ingredient of the present invention, in addition to the processed product derived from Peucedanum japonicum mentioned above, 3′-acetoxy-4′-senecioyloxy-3′,4′-dihydroseselin which is a kind of coumarins derived from Peucedanum japonicum, a derivative thereof, and/or pharmaceutically acceptable salts thereof can be used.

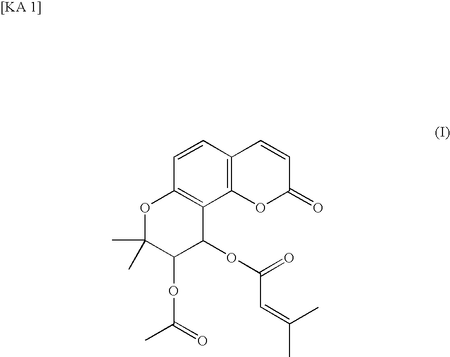

A method for preparing the above 3′-acetoxy-4′-senecioyloxy-3′,4′-dihydroseselin is not particularly limited. For example, 3′-acetoxy-4′-senecioyloxy-3′,4′-dihydroseselin can be obtained from an ethanol extract of Peucedanum japonicum by carrying out various kinds of chromatographies. Alternatively, in the case of synthesis, 3′-acetoxy-4′-senecioyloxy-3′,4′-dihydroseselin can be also obtained by a combination of known methods.

As the derivative as used herein, for example, a derivative (prodrug) which can be easily hydrolyzed in a body to form the above-mentioned 3′-acetoxy-4′-senecioyloxy-3′,4′-dihydroseselin and exhibit the desired effects, such as an ester can be prepared. The prodrug may be prepared in accordance with a known process. In addition, the derivative of the present invention encompasses, for example, a derivative obtained by administering the compound of the present invention to a mammal, to give a product via metabolism. Here, the derivative may be a salt of 3′-acetoxy-4′-senecioyloxy-3′,4′-dihydroseselin.

As the salt of 3′-acetoxy-4′-senecioyloxy-3′,4′-dihydroseselin used in the present invention or a derivative thereof, a pharmaceutically acceptable salt is preferable. Alternatively, as mentioned above, the salt may be a derivative of the compound which can function as a prodrug. Therefore, 3′-acetoxy-4′-senecioyloxy-3′,4′-dihydroseselin of the present invention encompasses a derivative thereof and salts thereof, as long as the desired effects of the present invention can be obtained. In addition, various isomers such as an optical isomer, a keto-enol tautomer, and a geometrical isomer of 3′-acetoxy-4′-senecioyloxy-3′,4′-dihydroseselin, and isolated products of each of isomers can be all used in the present invention as long as these isomers have an anti-foaming action for a cell or an inhibitory action for ACAT.

The salt of the compounds used in the present invention is exemplified by, for example, alkali metal salts, alkaline earth metal salts, salts with organic bases, and the like. The pharmaceutically acceptable salt used in the present invention means a salt of a compound which is substantially atoxic to an organism and has an anti-foaming action for a cell. The salt includes, for example, salts with sodium, potassium, calcium, magnesium, ammonium, protonated benzathine (N,N′-di-benzylethylenediamine), choline, ethanolamine, diethanolamine, ethylenediamine, meglamine (N-methylglucamine), benethamine (N-benzylphenethylamine), piperazine, tolomethamine (2-amino-2-hydroxymethyl-1,3-propanediol) or the like.

In the present invention, the processed product of the present invention, and 3′-acetoxy-4′-senecioyloxy-3′,4′-dihydroseselin, a derivative thereof and/or pharmaceutically acceptable salts thereof are referred to as the effective ingredient of the present invention, and a therapeutic agent or prophylactic agent for a disease requiring an anti-foaming action for a cell and/or an inhibitory action for ACAT, which comprises the effective ingredient of the present invention may be referred to as the therapeutic agent or prophylactic agent of the present invention in some cases. Also, in the case of the medicament of the present invention, the medicament encompasses an anti-foaming agent for a cell and an inhibitory agent for ACAT set forth below in addition to the therapeutic agent and the prophylactic agent in some cases.

The effective ingredient of the present invention is derived from Peucedanum japonicum, which has been used for edible purposes since ancient times and has a higher safety as compared to a synthetic compound having an inhibitory action for ACAT which has been known so far (for example, FR179254 set forth below). No toxicity is especially found in the effective ingredient of the present invention as mentioned later, and there is no risk of the onset of side effects. For these reasons, the disease can be safely and appropriately treated or prevented. Therefore, the therapeutic agent, the prophylactic agent, the food, or the feed, each comprising the effective ingredient, is effective in treating or preventing a disease requiring an anti-foaming action for a cell and an inhibitory action for ACAT, and is especially useful as a functional food material which is conveniently ingestible.

In addition, in the present invention, a disease requiring an anti-foaming action for a cell in treatment or prevention is not particularly limited as long as a disease in which an effect of treatment or prevention can be seen by suppressing foaming for a cell, and is exemplified by, for example, arteriosclerosis and a disease caused by arteriosclerosis as a causal factor, for example, ischemic heart disease, acute myocardial infarction, unstable angina, ischemic sudden death, cerebrovascular accident, chronic obstructive arteriosclerosis, myocardial infarction, angina pectoris, cerebral infarction, subarachnoid hemorrhage, obesity, and the like (for example, see O'Rourke et al., J. Biol. Chem., 2002, 277(45), 42557-42562).

In addition, a disease requiring an inhibitory action for ACAT in treatment or prevention in the present invention is not particularly limited and is exemplified by, in addition to the above-mentioned disease requiring an anti-foaming action for a cell in treatment or prevention, hyperlipemia, hypercholesterolemia, hypertriglyceridemia, multiple risk factor syndrome, and a disease caused by these diseases as a causal factor (for example, see O'Rourke et al., J. Biol. Chem., 2002, 277(45), 42557-42562, Ohishi et al., Biol. Pharm. Bull., 2003, 26(8), 1125-1128, and Ohishi et al., Chem. Pharm. Bull., 2001, 49(7), 830-839).

The therapeutic agent or prophylactic agent of the present invention includes ones formed into a preparation by combining the above-mentioned effective ingredient according to the present invention with a known pharmaceutical carrier. Also, as the therapeutic agent or prophylactic agent of the present invention, the above-mentioned effective ingredient can be combined with other component which can be used for the same applications as those of the effective ingredients, for example, a known component having an action of treating or preventing hyperlipemia or arteriosclerosis, for example, an inhibitory agent for HMG-CoA reductase such as a statin-based compound such as pravastatin, simvastatin, fluvastatin, cerivastatin, or atorvastatin, an anti-foaming agent such as fucoidan, an inhibitory agent for ACAT such as melinamide, a suppressive agent for the cholesterol ester transfer protein (CETP), an inhibitory agent for cholesterol absorption, an inhibitory agent for an enzyme synthesizing squalene, a suppressive agent for LDL oxidization, an inhibitory agent for microsomal triglyceride transfer protein (MTP), an agent for enhancing apolipoprotein A1 production, an agent for inducing ATP-binding cassette subfamily A1 (ABCA1), an inhibitory agent for squalene epoxidase, a bile acid binding resin such as cholestyramine, fibrate-based such as clofibrate, nicotinic acid-based such as niacin, an agent for lowering triglyceride such as ethyl eicosapentaenoate or the like.

The therapeutic agent or prophylactic agent of the present invention is usually manufactured by combining the above-mentioned effective ingredient with a pharmaceutically acceptable liquid or solid carrier. A solvent, a dispersant, an emulsifier, a buffer, a stabilizer, an excipient, a binder, a disintegrant, a lubricant, or the like can be optionally added thereto, to form a solid agent such as a tablet, a granule, a powder, an epipastic, and a capsule, or a liquid agent such as a common liquid agent, a suspension agent or an emulsion agent. In addition, there can be also formed into a dry product which can be liquefied by adding an appropriate carrier before use, or also into an external preparation.

The pharmaceutical carrier can be selected depending upon the administration form and preparation form of the therapeutic agent or prophylactic agent. In the case of an orally administered preparation comprising a solid composition, the preparation can be produced in the form of a tablet, a pill, a capsule, a powder, a fine powder, a granule or the like, and there can be utilized a pharmaceutical carrier such as starch, lactose, saccharose, mannitol, carboxymethyl cellulose, cornstarch, or an inorganic salt. In addition, upon preparation of the orally administered preparation, a binder, a disintegrant, a surfactant, a lubricant, a fluidity accelerator, a corrective, a colorant, a flavor, and the like can be further combined therewith. In the case of forming into a tablet or a pill, for example, the tablet or pill may be covered with a sugar-coating made of sucrose, gelatin or hydroxypropyl cellulose, or with a film made of a substance soluble in the stomach or intestine as desired. In the case of an orally administered preparation comprising a liquid composition, the preparation can be prepared in the form of a pharmaceutically acceptable emulsion, solution, suspension, syrup, or the like. In this case, for example, purified water, ethanol or the like is utilized as a carrier. Furthermore, an auxiliary agent such as a wetting agent or a suspending agent, a sweetener, a flavor, an antiseptic, or the like may be added as desired.

On the other hand, in the case of a parenterally administered preparation, the preparation can be prepared by dissolving or suspending the above-mentioned effective ingredient of the present invention in a diluent such as distilled water for injection, physiological saline, an aqueous solution of glucose, vegetable oil for injection, sesame oil, peanut oil, soybean oil, corn oil, propylene glycol or polyethylene glycol, in accordance with a conventional method, and adding a microbicide, a stabilizer, a tonicity agent, a soothing agent, or the like if needed. It is also possible to produce a solid composition and dissolve the composition in sterile water or a sterile solvent for injection before use.

The external preparation includes solid, semi-solid or liquid preparations for percutaneous administration or transmucosal (intraoral or intranasal) administration. The external preparation also includes suppositories and the like. For example, the external preparation may be prepared as liquid preparations including emulsions, suspensions such as lotions, external tinctures, and liquid agents for transmucosal administration; ointments such as oily ointments and hydrophilic ointments; patches for percutaneous administration or transmucosal administration such as films, tapes and poultices; and the like.

The therapeutic agent or prophylactic agent of the above-mentioned various preparation forms can be appropriately produced in accordance with conventional methods by utilizing known pharmaceutical carriers and the like. Also, the content of the effective ingredient in the therapeutic agent or prophylactic agent is not particularly limited, as long as the content is preferably in an amount so that the effective ingredient can be administered within the dose range described below in consideration of administration form, administration method and the like of the preparation. The content of the effective ingredient in the medicament of the present invention is usually from 1 to 100% by weight or so.

The therapeutic agent or prophylactic agent of the present invention is administered via an administration route appropriate for each of the preparation form. The administration method is also not limited to specific one. The agent can be administered internally, externally and by injection. In a case where the therapeutic agent or prophylactic agent of the present invention is administered by the injection, the agent can be administered, for example, intravenously, intramuscularly, subcutaneously, intracutaneously, or the like. In a case where the agent is externally administered, for example, a suppository or the like as an external preparation may be administered according to its proper administration method.

The dose of the therapeutic agent or prophylactic agent of the present invention is changeable and properly set depending upon its preparation form, administration method, purpose of use, and age, weight, symptom or the like of a patient to which the therapeutic agent or prophylactic agent is administered, or the like. Generally, the dose of the agent, in terms of the dose of the above-mentioned effective ingredient contained in the preparation, is, for example, in a case where the processed product of the present invention is used as the effective ingredient, preferably from 0.1 μg to 10 g/kg weight, more preferably from 1 μg to 5 g/kg weight, and even more preferably from 10 μg to 1 g/kg weight, per day for adult, and in a case where 3′-acetoxy-4′-senecioyloxy-3′,4′-dihydroseselin, a derivative thereof and/or pharmaceutically acceptable salts thereof is used as the effective ingredient, preferably from 0.1 μg to 5 g/kg weight, more preferably from 1 μg to 2 g/kg weight, and even more preferably from 10 μg to 1 g/kg weight, per day for adult. As a matter of course, the dose varies depending upon various conditions, so that an amount smaller than the dose mentioned above may be sufficient, or an amount exceeding the dose range may be required. Administration may be carried out once or in several divided portions in a day within the desired dose range. The administration period may be arbitrarily determined. Also, the therapeutic agent or prophylactic agent of the present invention can be directly orally administered, or the agent can be added to any food to be taken on a daily basis.

In addition, the present invention provides an anti-foaming agent for a cell comprising the above-mentioned effective ingredient. The anti-foaming agent may be the above-mentioned effective ingredient itself, or a composition comprising the above-mentioned effective ingredient. The anti-foaming agent can be also prepared by combining the above-mentioned effective ingredient with other component which can be used for the same applications as those of the effective ingredients, for example, a known component having an action of treating or preventing arteriosclerosis, for example, an inhibitory agent for HMG-CoA reductase such as a statin-based compound such as pravastatin, simvastatin, fluvastatin, cerivastatin, or atorvastatin, an anti-foaming agent such as fucoidan, an inhibitory agent for ACAT such as melinamide, a suppressive agent for the cholesterol ester transfer protein (CETP), an inhibitory agent for cholesterol absorption, an inhibitory agent for an enzyme synthesizing squalene, a suppressive agent for LDL oxidization, an inhibitory agent for microsomal triglyceride transfer protein (MTP), an agent for, enhancing apolipoprotein A1 production, an agent for inducing ATP-binding cassette subfamily A1 (ABCA1), an inhibitory agent for squalene epoxidase, a bile acid binding resin such as cholestyramine, fibrate-based such as clofibrate, nicotinic acid-based such as niacin, an agent for lowering triglyceride such as ethyl eicosapentaenoate or the like. The anti-foaming agent can be also prepared into a form of reagent usually used according to the above-mentioned process for preparing the therapeutic agent or prophylactic agent. The content of the above-mentioned effective ingredient in the anti-foaming agent is not particularly limited, as long as the content is in an amount so that the desired effects of the present invention can be exhibited in consideration of administration method, purpose of use or the like of the anti-foaming agent. The content of the effective ingredient in the anti-foaming agent of the present invention is usually from 1 to 100% by weight or so. Also, the amount of the anti-foaming agent used is not particularly limited, as long as the desired effects of the present invention can be exhibited. Especially in the case where the anti-foaming agent is administered to a living body, the anti-foaming agent may be preferably used in an amount so that the effective ingredient can be administered within the dose range of the effective ingredient for the above-mentioned therapeutic agent or prophylactic agent. The administration method is also not particularly limited, and may be properly set in the same manner as the above-mentioned therapeutic agent or prophylactic agent. The anti-foaming agent is useful in treating or preventing a disease requiring an anti-foaming action for a cell in treatment or prevention, for example, arteriosclerosis, and a disease caused by arteriosclerosis as a causal factor. In addition, the anti-foaming agent is useful in screening of drugs for a disease requiring an anti-foaming action for a cell in treatment or prevention. Furthermore, the anti-foaming agent is useful in studies on mechanisms of foaming for arteriosclerosis or various cells, or functional studies relating to physical changes in the cells. In addition, the anti-foaming agent can be added to food or beverage.

In addition, the present invention provides an inhibitory agent for ACAT comprising the above-mentioned effective ingredient. The inhibitory agent for ACAT may be the above-mentioned effective ingredient itself, or a composition comprising the above-mentioned effective ingredient. The inhibitory agent for ACAT can be also prepared by combining the above-mentioned effective ingredient with other component which can be used for the same applications as those of the effective ingredients, for example, a known component having an action of treating or preventing hyperlipemia or arteriosclerosis, for example, an inhibitory agent for HMG-CoA reductase such as a statin-based compound such as pravastatin, simvastatin, fluvastatin, cerivastatin, or atorvastatin, an anti-foaming agent such as fucoidan, an inhibitory agent for ACAT such as melinamide, a suppressive agent for the cholesterol ester transfer protein (CETP), an inhibitory agent for cholesterol absorption, an inhibitory agent for an enzyme synthesizing squalene, a suppressive agent for LDL oxidization, an inhibitory agent for microsomal triglyceride transfer protein (MTP), an agent for enhancing apolipoprotein A1 production, an agent for inducing ATT-binding cassette subfamily A1 (ABCA1), an inhibitory agent for squalene epoxidase, a bile acid binding resin such as cholestyramine, fibrate-based such as clofibrate, nicotinic acid-based such as niacin, an agent for lowering triglyceride such as ethyl eicosapentaenoate or the like. The inhibitory agent for ACAT can be also prepared into a form of reagent usually used according to the above-mentioned process for preparing the therapeutic agent or prophylactic agent. The content of the above-mentioned effective ingredient in the inhibitory agent for ACAT is not particularly limited, as long as the content is in an amount so that the desired effects of the present invention can be exhibited in consideration of administration method, purpose of use or the like of the inhibitory agent for ACAT. The content of the effective ingredient in the inhibitory agent for ACAT of the present invention is usually from 1 to 100% by weight or so. Also, the amount of the inhibitory agent for ACAT used is not particularly limited, as long as the desired effects of the present invention can be exhibited. Especially in the case where the inhibitory agent for ACAT is administered to a living body, the inhibitory agent for ACAT may be preferably used in an amount so that the effective ingredient can be administered within the dose range of the effective ingredient for the above-mentioned therapeutic agent or prophylactic agent. The administration method is also not particularly limited, and may be properly set in the same manner as the above-mentioned therapeutic agent or prophylactic agent. The inhibitory agent for ACAT is useful in treating or preventing a disease requiring an inhibitory action for ACAT in treatment or prevention, for example, arteriosclerosis, hyperlipemia, and a disease caused by these diseases as a causal factor. In addition, the inhibitory agent for ACAT is useful in screening of drugs for a disease requiring an inhibitory action for ACAT in treatment or prevention. Furthermore, the inhibitory agent for ACAT is useful in studies on mechanisms of the cholesterol ester production, and also in studies on mechanisms relating to the progression of the above-mentioned diseases such as hyperlipemia and arteriosclerosis by the cholesterol ester. In addition, the inhibitory agent for ACAT can be added to food or beverage.

No toxicity is especially found in the effective ingredient of the present invention as described later. Also, there is no risk of the onset of side effects. For these reasons, the anti-foaming action for a cell and/or the inhibitory action for ACAT can be safely and appropriately exhibited in a living body. Therefore, the medicament, food or feed of the present invention comprising the effective ingredient is effective in treating or preventing a disease requiring an anti-foaming action for a cell and/or an inhibitory action for ACAT in treatment or prevention.

In addition, the present invention provides a food or feed for anti-foaming for a cell and/or an inhibition for ACAT, comprising the above-mentioned effective ingredient of the present invention (referred herein as the food or feed of the present invention in some cases). Since the food or feed of the present invention has an anti-foaming action for a cell and/or an inhibitory action for ACAT, the food or feed is very useful in amelioration of symptoms or prevention of a disease requiring an anti-foaming action for a cell and/or an inhibitory action for ACAT in treatment or prevention, more specifically, arteriosclerosis, ischemic heart disease, acute myocardial infarction, unstable angina, ischemic sudden death, cerebrovascular accident, chronic obstructive arteriosclerosis, hypercholesterolemia, hypertriglyceridemia, hyperlipemia, multiple risk factor syndrome, as mentioned above. More specifically, the food or beverage of the present invention is very useful as a functional food (food for specified health use or the like) showing that is intended to prevent, ameliorate, or treat the above-mentioned disease, so that the food or beverage is very useful for an individual who cares about his/her blood cholesterol level, an individual who cares about his/her triglyceride, or an individual who cares about his/her body fat.

In the food or feed of the present invention, the effective ingredient of the present invention is combined with other substance having an action of ameliorating, treating, or preventing hyperlipemia or arteriosclerosis, for example, a known component having an action of treating or preventing hyperlipemia or arteriosclerosis, for example, an inhibitory agent for HMG-CoA reductase such as a statin-based compound such as pravastatin, simvastatin, fluvastatin, cerivastatin, or atorvastatin, an anti-foaming agent such as fucoidan, an inhibitory agent for ACAT such as melinamide, a suppressive agent for the cholesterol ester transfer protein (CETP), an inhibitory agent for cholesterol absorption, an inhibitory agent for an enzyme synthesizing squalene, a suppressive agent for LDL oxidization, an inhibitory agent for microsomal triglyceride transfer protein (MTP), an agent for enhancing apolipoprotein A1 production, an agent for inducing ATP-binding cassette subfamily A1 (ABCA1), an inhibitory agent for squalene epoxidase, a bile acid binding resin such as cholestyramine, fibrate-based such as clofibrate, nicotinic acid-based such as niacin, an agent for lowering triglyceride such as ethyl eicosapentaenoate or the like, and whereby the more effective food or feed can be also prepared. Alternatively, the effective ingredient of the present invention can be combined with a known functional food material, for example, Angelica keiskei koidz or a processed product thereof, peptide, glucomannan, chitosan, a plant sterol ester, EPA, DHA or the like.

The term “comprising” in the food or feed of the present invention encompasses the meanings of containing(ed), adding(ed) and/or diluting(ed). As used herein, the term “containing(ed)” refers to an embodiment of containing the effective ingredient usable in the present invention in the food or feed; the term “adding(ed)” refers to an embodiment of adding the effective ingredient usable in the present invention to a raw material for the food or feed; and the term “diluting(ed)” refers to an embodiment of adding a raw material for the food or feed to the effective ingredient usable in the present invention.

The process for preparing the food or feed of the present invention is not particularly limited. For example, combination, cooking, processing, and the like can be carried out in accordance with those generally employed for foods or feeds, and the food or feed of the present invention can be prepared by the general methods for preparing a food or feed, as long as the resulting food or feed contains the above-mentioned effective ingredient of the present invention having an anti-foaming action for a cell and/or an inhibitory action for ACAT.

The food of the present invention is not particularly limited. The food includes, for example, processed agricultural and forest products, processed livestock products, processed marine products and the like, including processed grain products such as processed wheat products, processed starch products, processed premix products, noodles, macaronis, bread, bean jam, buckwheat noodles, wheat-gluten bread, rice noodles, fen-tiao, and packed rice cake; processed fat and oil products such as plastic fat and oil, tempura oil, salad oil, mayonnaise, and dressing; processed soybean products such as tofu products, soybean paste (miso), and fermented soybeans; processed meat products such as ham, bacon, pressed ham, and sausage; marine products such as frozen ground fish, steamed fish paste, tubular roll of steamed fish paste, cake of ground fish, deep-fried patty of fish paste, fish ball, fascia and tendon, fish meat ham, sausage, dried bonito, products of processed fish egg, canned marine products, and preserved food boiled down in soy sauce (tsukudani); milk products such as raw material milk, cream, yoghurt, butter, cheese, condensed milk, powder milk, and ice cream; processed vegetable and fruit products such as paste, jam, pickled vegetables, fruit beverages, vegetable beverages, and mixed beverages; confectioneries such as chocolates, biscuits, sweet bun, cake, rice cake snacks and rice snacks; alcohol beverages such as sake, Chinese liquor, wine, whiskey, Japanese distilled liquor (shochu), vodka, brandy, gin, rum, beer, refreshing alcoholic beverages, fruit liquor, and liqueur; luxury drinks such as green tea, tea, oolong tea, coffee, refreshing beverages and lactic acid beverages; seasonings such as soy sauce, sauce, vinegar, and sweet rice wine; canned, bottled or pouched foods such as rice topped with cooked beef and vegetable, rice boiled together with meat and vegetables in a small pot, steamed rice with red beans, curry roux and rice, and other precooked foods; semi-dry or concentrated foods such as liver pastes and other spreads, soups for buckwheat noodles or wheat noodles, and concentrated soups; dry foods such as instant noodles, instant curry roux, instant coffee, powder juice, powder soup, instant soybean paste (miso) soup, precooked foods, precooked beverages, and precooked soup; frozen foods such as sukiyaki, pot-steamed hotchpotch, split and grilled eel, hamburger steak, shao-mai, dumpling stuffed with minced pork, various sticks, and fruit cocktails; solid foods; liquid foods such as soups; spices; and the like, in which each of the foods comprises the above-mentioned effective ingredient of the present invention. Here, the food also encompasses the beverage herein.

In the food of the present invention, the above-mentioned effective ingredient of the present invention is contained, added and/or diluted, alone or in plurality, and its shape is not particularly limited, as long as the content corresponds to an amount required to exhibit an anti-foaming action for a cell and/or an inhibitory action for ACAT. The shape includes those which are orally ingestible such as powders, tablets, granules and capsules. In addition, the food of the present invention encompasses a processed product derived from Peucedanum japonicum as it is, or a product obtained by properly mixing the processed product with an appropriate emulsifying agent, an excipient, or the like. These foods can be eaten as beverages as they are or as mixed with water.

In addition, as to the food of the present invention, there can be prepared into healthcare drink by mixing the effective ingredient of the present invention with a squeezed juice of a plant other than those plants usable in the present invention, for example, a vegetable, a fruit or the like, or squeezing the plant together with the plant usable in the present invention. For example, the healthcare drink having an anti-foaming action for a cell and/or an inhibitory action for ACAT can be prepared by diluting a squeezed juice of various plants used in the present invention with water, or mixing the squeezed juice with a squeezed juice of Angelica keiskei koidz., parsley, celery, licorice, carrot, Brassica Rapa var. pervidis (komatsuna), turnip, pak-choi, tomato, mandarin orange, lemon, grapefruit, kiwi, spinach, radish, Japanese radish (daikon), celery cabbage, cabbage, Butter head lettuce, lettuce, Chinese chive, okra, green pepper, cucumber, kidney beans, green soybeans, common pea, Indian corn, Rocket, arugula, loquat, Citrus natsudaidai, amanatsu, or the like, cow's milk, soybean milk or the like.

The content of the above-mentioned effective ingredient in the food of the present invention is not particularly limited, and the content can be appropriately selected from the viewpoint of sensory aspect and exhibition of activity. For example, in the case where the processed product of the present invention is used as the effective ingredient, the content is preferably 0.1% by weight or more, more preferably from 0.5 to 95% by weight, and even more preferably from 1 to 90% by weight, of the food. Alternatively, in the case where 3′-acetoxy-4′-senecioyloxy-3′,4′-dihydroseselin, a derivative thereof, and/or pharmaceutically acceptable salts thereof is used as the effective ingredient, the content is preferably 0.00001% by weight or more, more preferably from 0.0001 to 10% by weight, and even more preferably from 0.0006 to 6% by weight, of the food.

Also, for example, in the case where the processed product of the present invention is used as the effective ingredient, the food of the present invention may be taken so that the effective ingredient contained therein is taken in an amount of preferably from 0.1 μg to 10 g/kg weight, more preferably from 1 μg to 5 g/kg weight, and even more preferably from 10 μg to 2 g/kg weight, per day for adult. Alternatively, in the case where 3′-acetoxy-4′-senecioyloxy-3′,4′-dihydroseselin, a derivative thereof, and/or pharmaceutically acceptable salts thereof is used as the effective ingredient, the food of the present invention may be taken so that the effective ingredient contained therein is taken in an amount of preferably from 0.1 μg to 5 g/kg weight, more preferably from 1 μg to 2 g/kg weight, and even more preferably from 10 μg to 1 g/kg weight, per day for adult.

In addition, the present invention provides a feed for an organism having an anti-foaming action for a cell and/or an inhibitory action for ACAT, in which the above-mentioned effective ingredient is comprised, more specifically, contained, added and/or diluted. In still another embodiment, the present invention also provides a method of feeding an organism, characterized by administering the above-mentioned effective ingredient to the organism. In still yet another embodiment, the present invention provides an organism-feeding agent characterized in that the organism-feeding agent comprises the above-mentioned effective ingredient.

The organism as used herein is not limited, and includes, for example, the cultured or bred animals, pet animals, and the like. The cultured or bred animal is exemplified by livestock such as Equus, Bos, Porcus, Ovens, Capra, Camelus, and Lama; laboratory animals such as mice, rats, guinea pigs, and rabbits; poultry such as Chrysolophus, ducks, Meleagris, and Struthioniformes; pisces, crustacea or shellfish. The pet animal is exemplified by dogs, cats, and the like. The feed is exemplified by a feed for sustenance of and/or amelioration in physical conditions. The organism-feeding agent is exemplified by immersion agents, feed additives, and beverage additives.

According to these inventions, the same effects can be expected to be exhibited as those of the above-mentioned therapeutic agent or prophylactic agent of the present invention, on the basis of an anti-foaming action for a cell and/or an inhibitory action for ACAT of the above-mentioned effective ingredient used in the present invention, in the organism exemplified above to which these are applied. In other words, the feed of the present invention can exhibit an effect for treating or preventing a disease requiring an anti-foaming action for a cell and/or an inhibitory action for ACAT in treatment or prevention, for example, arteriosclerosis, hyperlipemia, or a disease caused by these diseases as a causal factor.

For example, in the case where the processed product of the present invention is used as the effective ingredient, the above-mentioned effective ingredient used in the present invention is usually administered so that the effective ingredient contained therein is in an amount of preferably from 0.1 μg to 10 g/kg weight, more preferably from 1 μg to 5 g/kg weight, and even more preferably from 10 μg to 2 g/kg weight, per day for a subject organism. Alternatively, in the case where 3′-acetoxy-4′-senecioyloxy-3′,4′-dihydroseselin, a derivative thereof, and/or pharmaceutically acceptable salts thereof is used as the effective ingredient, the above-mentioned effective ingredient used in the present invention is usually administered so that the effective ingredient contained therein is in an amount of preferably from 0.1 μg to 5 g/kg weight, more preferably from 1 μg to 2 g/kg weight, and even more preferably from 10 μg to 1 g/kg weight, per day for a subject organism. The administration can be carried out, for example, by previously adding and mixing the effective ingredient in a raw material for an artificially formulated feed to be given to a subject organism, or mixing the effective ingredient with a powder raw material for an artificially formulated feed, and thereafter further adding and mixing the mixture with other raw materials. The content of the above-mentioned effective ingredient in the feed is not particularly limited, and can be appropriately set in accordance with the purposes. For example, in the case where the processed product of the present invention is used as the effective ingredient, the above-mentioned effective ingredient is contained in the feed in an amount of preferably from 0.1% by weight or more, more preferably from 0.5 to 95% by weight, and even more preferably from 1 to 90% by weight. Alternatively, in the case where 3′-acetoxy-4′-senecioyloxy-3′,4′-dihydroseselin, a derivative thereof, and/or pharmaceutically acceptable salts thereof is used as the effective ingredient, the above-mentioned effective ingredient is contained in the feed in an amount of preferably from 0.00001% by weight or more, more preferably from 0.0001 to 10% by weight, and even more preferably from 0.0006 to 6% by weight. The content of the effective ingredient of the present invention in the organism-feeding agent may be adjusted to the same level as the feed.

The process for preparing the feed of the present invention is not particularly limited, and its composition may be set in accordance with a general feed, as long as the above-mentioned effective ingredient according to the present invention having an anti-foaming action for a cell and/or an inhibitory action for ACAT is contained in the feed prepared. The organism-feeding agent can also be prepared in the same manner.

In the present invention, for example, by allowing a subject organism to take the feed comprising the above-mentioned effective ingredient used in the present invention having an anti-foaming action for a cell and/or an inhibitory action for ACAT, or immersing a subject organism into a solution containing the above-mentioned effective ingredient used in the present invention having an anti-foaming action for a cell (for example, prepared by dissolving the above-mentioned immersing agent in water), the physical conditions of the livestock, laboratory animals, poultry, pet animals or the like can be well sustained or ameliorated. These embodiments are one embodiment of the feeding method of an organism in the present invention.

The present invention also provides a method of treating or preventing a disease requiring an anti-foaming action for a cell and/or an inhibitory action for ACAT in treatment or prevention, comprising administering to a subject the above-mentioned effective ingredient.

The term “subject” as used herein is preferably a human in need of an anti-foaming action for a cell and/or an inhibitory action for ACAT, and may include the cultured or bred animals, pet animals, and the like as mentioned above.

In addition, the term “effective amount” as used herein is, in the case where the above-mentioned effective ingredient has been administered to the above-mentioned subject, an amount of the ingredient exhibiting an anti-foaming action for a cell and/or an inhibitory action for ACAT as compared to a subject to which the effective ingredient has not been administered. The specific effective amount is changeable and properly set depending upon its administration form, administration method, purpose of use, and age, weight, symptom or the like of a subject. Preferably, as same as the above-mentioned medicament, in the case where the processed product of the present invention is used as the effective ingredient, the processed product is administered so that the effective ingredient contained therein is in an amount of preferably from 0.1 μg to 10 g/kg weight, more preferably from 1 μg to 5 g/kg weight, and even more preferably from 10 μg to 2 g/kg weight, per day for a human (for example, adult). Alternatively, in the case where 3′-acetoxy-4′-senecioyloxy-3′,4′-dihydroseselin, a derivative thereof, and/or pharmaceutically acceptable salts thereof is used as the effective ingredient, the processed product is administered so that the effective ingredient contained therein is in an amount of preferably from 0.1 μg to 5 g/kg weight, more preferably from 1 μg to 2 g/kg weight, and even more preferably from 10 μg to 1 g/kg weight, per day for a human (for example, adult).

In a method of treating or preventing a disease requiring an anti-foaming action for a cell and/or an inhibitory action for ACAT in treatment or prevention of the present invention, an effective amount of the above-mentioned effective ingredient can be directly administered to the above-mentioned subject, and can be administered as the above-mentioned medicament, food, or feed. In addition, the administration method is not limited to specific one, and for example, as same as the above-mentioned medicament, the above-mentioned effective ingredient can be administered orally, by injection, or the like.

According to the method of treatment or prevention of the present invention, a disease which is a target of the above-mentioned medicament, food or feed of the present invention can be treated or prevented. For example, an effect for treating or preventing arteriosclerosis, hyperlipemia, or a disease caused by these diseases as a causal factor can be exhibited.

No toxicity is found even when the above-mentioned effective ingredient used in the present invention is administered in an effective dose for the exhibition of its action in a living body. For example, in the case of oral administration, no cases of deaths are found even when an ethanol extract of Peucedanum japonicum or 3′-acetoxy-4′-senecioyloxy-3′,4′-dihydroseselin, is administered to a mouse at each 1 g/kg weight in a single dose. In addition, no cases of deaths are found even when the above-mentioned effective ingredient is orally administered to a rat at 1 g/kg weight in a single dose.

### EXAMPLES

The present invention will be described more concretely hereinbelow by means of the examples, but the present invention is by no means limited to these descriptions. Unless specified otherwise, % in Examples all means % by volume.

### Preparation Example 1

### Preparation of Ethanol Extract Fraction from Stem of Peucedanum japonicum and 70% Ethanol Extract Fraction from Stem of Peucedanum japonicum

Forty milliliters of ethanol or a 70% ethanol was added to 2 g of a product prepared by powdering a lyophilized product of a stem portion of Peucedanum japonicum, and extraction was carried out at room temperature for 30 minutes. The mixture was separated into an extract and a residue by centrifugation. Subsequently, the extraction procedures with 30 ml of the same solvent were repeated twice for the residue. The obtained extracts were combined, and the combined extract was concentrated with a rotary evaporator. Finally, an ethanol extract fraction was dissolved in 1 ml of dimethyl sulfoxide, and a 70% ethanol extract fraction was dissolved in 2 ml of dimethyl sulfoxide, to give an ethanol extract fraction from a stem of Peucedanum japonicum and a 70% ethanol extract fraction from a stem of Peucedanum japonica, respectively.

### Preparation Example 2

### Preparation of Ethanol Extract Fraction from Root of Peucedanum japonicum

Forty milliliters of ethanol was added to 2 g of a product prepared by powdering a lyophilized product of a root portion of Peucedanum japonicum, and extraction was carried out at room temperature for 30 minutes. The mixture was separated into an extract and a residue by centrifugation. Subsequently, the extraction procedures with 30 ml of the same solvent were repeated twice for the residue. The obtained extracts were combined, and the combined extract was concentrated with a rotary evaporator. Finally, an ethanol extract fraction was dissolved in 1 ml of dimethyl sulfoxide, to give an ethanol extract fraction from a root of Peucedanum japonicum.

### Preparation Example 3

### Preparation of Ethanol Extract Fraction from Leaf of Peucedanum japonicum

Forty milliliters of ethanol was added to 2 g of a product prepared by powdering a lyophilized product of a leaf portion of Peucedanum japonicum, and extraction was carried out at room temperature for 30 minutes. The mixture was separated into an extract and a residue by centrifugation. Subsequently, the extraction procedures with 30 ml of the same solvent were repeated twice for the residue. The obtained extracts were combined, and the combined extract was concentrated with a rotary evaporator. Finally, an ethanol extract fraction was dissolved in 1 ml of dimethyl sulfoxide, to give an ethanol extract fraction from a leaf of Peucedanum japonicum.

### Preparation Example 4

### Fractionation with Reverse Phase Column of Ethanol Extract Fraction from Leaf of Peucedanum japonicum

(1) Two-hundred milliliters of ethanol was added to 10 g of a product prepared by powdering a lyophilized product of a leaf portion of Peucedanum japonicum, and extraction was carried out at room temperature for 30 minutes. The mixture was separated into an extract and a residue by suction filtration. Subsequently, the extraction procedures with 150 ml of the same solvent were repeated twice for the residue. The obtained extracts were combined, and the combined extract was concentrated with a rotary evaporator. Finally, an ethanol extract fraction was dissolved in 5 ml of dimethyl sulfoxide, to give an ethanol extract from a leaf of Peucedanum japonicum.

(2) The ethanol extract from a leaf of Peucedanum japonicum obtained in item (1) of Preparation Example 4 was fractionated by using reverse phase chromatography. As the resin, Cosmosil 75 C18-OPN (manufactured by Nakalai Tesque, Inc.: 50 mL) equilibrated with a 20% ethanol was used. The ethanol extract from a leaf of Peucedanum japonicum was added to the resin, and thereafter the elution was carried out with 200 mL of a 20% ethanol, 600 mL of a 40% ethanol, and 400 mL of a 60% ethanol in that order.

(3) The eluate with the 20% ethanol, first 400 ml of the eluate with the 40% ethanol, and last 200 ml of the eluate with the 40% ethanol combined with the eluate with the 60% ethanol obtained in item (2) of Preparation Example 4 were each concentrated with a rotary evaporator. Finally, each of the ethanol eluates was dissolved in 5 ml of dimethyl sulfoxide, to give ethanol extract fractions from a leaf of Peucedanum japonicum, -Fr. 1, -Fr. 2, and -Fr. 3.

### Preparation Example 5

### Fractionation with Reverse Phase Column of Ethanol Extract Fraction from Leaf of Peucedanum japonicum -Fr. 2

(1) The amount of 0.9 ml out of the ethanol extract fraction from a leaf of Peucedanum japonicum -Fr. 2 obtained in item (3) of Preparation Example 4 was fractionated by using reverse phase chromatography. As the column, TSK gel ODS-80Ts (21.5 mm×30 cm: manufactured by Tosoh Corporation) was used. The solvent was distilled water:acetonitrile=30:70, the elution rate was 5 mL/minute, and the detection was carried out at 215 nm. The eluates were fractionated using ultraviolet absorption of the eluates as an index.

(2) Each of the fraction including a peak at a retention time of 23.7 minutes, 35.1 minutes, or 41.8 minutes obtained in item (1) of Preparation Example 5 was concentrated with a rotary evaporator. Finally, each of the ethanol eluates was dissolved in 0.9 ml of dimethyl sulfoxide, to give ethanol extract fractions from a leaf of Peucedanum japonicum, -Fr. 2-1, -2, and -3.

(3) A mass spectrum (MS) of the ethanol extract fraction from leaf of Peucedanum japonicum -Fr. 2-2 obtained in item (2) of Preparation Example 5 was determined with a mass spectrometer (API300, manufactured by Applied Biosystems/MDS Sciex). As a result, a signal of m/z 387 ([M+H]⁺) was detected. Subsequently, various kinds of NMR spectra of the ethanol extract fraction from a leaf of Peucedanum japonicum -Fr. 2-2 was determined with a nuclear magnetic resonance (NMR) spectrometer (Model AVANCE600, manufactured by Bruker BIOSPIN). As a result of determinations of the MS spectrum and various kinds of NMR spectra, the ethanol extract fraction from a leaf of Peucedanum japonicum -Fr. 2-2 was identified to be 3′-acetoxy-4′-senecioyloxy-3′,4′-dihydroseselin, shown in the above-mentioned formula (I).

### Example 1

### Determination of Anti-Foaming in Macrophage

Macrophage introduces a modified LDL (such as acetyl LDL (Ac-LDL) into a cell to synthesize cholesterol ester, thereby foaming. An anti-foaming activity for the macrophage with each subject sample was determined.

(1) Foaming for Macrophage

RAW 264.7 cells (ATCC TIB 71) were suspended in Dulbecco's modified Eagle's medium (manufactured by Sigma, D5796) containing 10% fetal bovine serum (manufactured by Cambrex Corporation), so as to have a concentration of 4×10⁵ cells/ml, and the suspension was put to each well of a 24-well microtiter plate in an amount of 1 ml per well. The cells were cultured at 37° C. overnight in the presence of 5% carbon dioxide gas. Next, the medium was exchanged with UltraCHO media (manufactured by Cambrex Corporation, B2724) and 2 μL of the dimethyl sulfoxide solution of each of the subject samples prepared in the above-mentioned Preparation Examples was added to each well so as to have a concentration shown in the following Table 1. Here, the purified ethanol extract fraction from a leaf of Peucedanum japonicum -Fr. 2-2 was used for 3′-acetoxy-4′-senecioyloxy-3′,4′-dihydroseselin in Table 1. Further, Ac-LDL (manufactured by Biomedical Technology, Inc., BT-906) at a final concentration of 20 μg/mL was added to each well, and each mixture was cultured for 24 hours. Here, there were set a group without addition of Ac-LDL and a group with addition of dimethyl sulfoxide as controls.

(2) Determination of Amount of Biosynthesis of Cholesterol Ester

The total amount of cholesterol in a cell and the amount of free cholesterol were determined to calculate the amount of cholesterol ester as an index for anti-foaming for macrophage.

After the termination of the culture, the medium was removed, and the cells were washed with a 0.3% (w/v) BSA (manufactured by Sigma, A-8022) containing phosphate buffered saline and further washed with a phosphate buffered saline. The amount 0.5 mL of a solvent of hexane:isopropanol=3:2 was added to the cells. The mixture was allowed to stand at room temperature for 30 minutes, and thereafter the supernatant was collected. The procedures were repeated again, and a total of 1 mL of the supernatant was concentrated to dryness. The precipitate was dissolved in 30 μL of isopropanol, and thereafter the total amount of cholesterol contained in 10 μL of the resulting solution was determined with Cholesterol E-Test (manufactured by Wako Pure Chemical Industries, Ltd., 439-17501) and the amount of free cholesterol was determined with Free Cholesterol E-Test (manufactured by Wako Pure Chemical Industries, Ltd., 435-35801). All of the determinations were carried out twice. The amount of biosynthesis of cholesterol ester was determined by subtracting the amount of free cholesterol from the total amount of cholesterol. The anti-foaming activity was calculated in accordance with the following formula.

Anti-Foaming Activity (%)=100−((Amount of Biosynthesis of Cholesterol Ester When Subject Sample Was Added Thereto)−(Amount of Biosynthesis of Cholesterol Ester of Group Without Addition of Ac-LDL))/((Amount of Biosynthesis of Cholesterol Ester When Dimethyl Sulfoxide Was Added Thereto)−(Amount of Biosynthesis of Cholesterol Ester of Group Without Addition of Ac-LDL))×100

The results are shown in Table 1. In other words, Table 1 shows an inhibitory activity for cholesterol ester accumulated in macrophage in each final concentration of each of the subject samples, and the remarkable anti-foaming activity was found in each sample described in the table.

TABLE 1

| | Final | |
|---|---|---|
| | Concentra- | Anti-Foaming |
| Subject Sample | tion (%) | Activity (%) |
| 70% Ethanol Extract Fraction from Stem | 0.2 | 78 |
| of Peucedanum japonicum | 0.1 | 33 |
| Ethanol Extract Fraction from Stem of | 0.05 | 86 |
| Peucedanum japonicum | 0.025 | 70 |
| Ethanol Extract Fraction from Root of | 0.0125 | 27 |
| Peucedanum japonicum | | |
| Ethanol Extract Fraction from Leaf of | 0.025 | 97 |
| Peucedanum japonicum | 0.0125 | 52 |
| Ethanol Extract Fraction from Leaf of | 0.1 | 88 |
| Peucedanum japonicum - Fr.2 | 0.05 | 34 |
| Ethanol Extract Fraction from Leaf of | 0.2 | 72 |
| Peucedanum japonicum - Fr.2-2 | 0.1 | 34 |
| Ethanol Extract Fraction from Leaf of | 0.2 | 35 |
| Peucedanum japonicum - Fr.2-3 | | |
| | Final | |
| | Concentra- | Anti-Foaming |
| Subject Sample | tion (μM) | Activity (%) |
| 3′-acetoxy-4′-senecioyloxy- | 40 | 98 |
| 3′,4′-dihydroseselin | 20 | 19 |

### Example 2

### (1) Preparation of Rat Microsome

Four of Sprague-Dawley rats were bred for one week and thereafter sacrificed, and the livers were immediately excised. The excised livers were shortly washed with cold brine, and thereafter immersed (25 mL/a rat) into sucrose buffer (0.3 M sucrose, 50 mM Tris-HCl, 1 mM EDTA, 50 mM NaCl, pH 7.4). The resulting livers were homogenized, and the procedures of centrifuging at 10,000×g for 30 minutes and removing the formed precipitates were repeated twice for the homogenized livers. The resulting supernatant was centrifuged at 105,000×g for 70 minutes, and the precipitates were collected and suspended in an appropriate amount of 100 mM phosphate buffer (pH 7.4). The suspension was again suspended in the phosphate buffer so as to have a protein concentration of 10 mg/ml, and EDTA was added thereto so as to have a final concentration of 1 mM. Thereafter, the suspension was subjected to frozen storage at −80° C. until being used.

(2) Determination of Inhibitory Activity for ACAT of Each Sample

The enzymatic activity of ACAT was determined according to the partially modified method of Lee et al. (Planta Med., 2004, 70, 678-679).

In a 1.5 mL Eppendorf tube, 1 μL of a dimethyl sulfoxide solution of each subject sample was added to a 92 μL of a reaction aqueous solution (0.05 M potassium phosphate, 1 mM dithiothreitol, 9 mg/mL fatty acid-free bovine serum albumin, 200 μg/mL cholesterol, pH 7.4) containing 1.5 μL of the rat liver microsome prepared in item (1) of Example 2 so as to have a final concentration described in Table 2, and the mixture was incubated at 37° C. for 30 minutes. Here, the purified ethanol extract fraction from a leaf of Peucedanum japonicum -Fr. 2-2 was used for 3′-acetoxy-4′-senecioyloxy-3′,4′-dihydroseselin in Table 2. In addition, there were set a group without addition of the compound and with only addition of the dimethyl sulfoxide solution as a control group and a group with addition of a compound FR179254 of which inhibitory activity for ACAT is known (a final concentration of 10 μM, manufactured by Merck & Co., Inc., 344235) as a positive control. Thereafter, the mixture solution of 2 μL of 0.05 mCi/mL Oleoyl Coenzyme A, [oleoyl-9,10-³H]-(manufactured by MORAVEK BIOCHEMICALS, INC., MT1649) and 5 μL of 0.2 mg/mL Oleoyl Coenzyme A (manufactured by ICN, 591-20521) was added to each tube and mixed, and the mixture was then incubated at 37° C. for 5 minutes. Thereafter, the reaction was stopped by adding 0.5 mL of a solution of isopropanol:heptane=4:1 to the each tube. The amount 0.2 mL of 0.1 M potassium phosphate solution (pH 7.4) and 0.3 mL of heptane were added to the each tube and stirred for 15 seconds, and the mixture was thereafter centrifuged for 15 seconds. The radioactivity was determined with a liquid scintillation counter LS6500 (manufactured by Beckmann) using 150 μL of the resulting supernatant with Ultima Gold (manufactured by PerkinElmer Life Sciences Inc., 6013329) as a scintillation cocktail. Each reaction was carried out twice. The inhibitory activity for ACAT (%) of each of the subject samples was calculated in accordance with the following formula setting an inhibitory ratio upon addition of 10 μM FR179254 which is a positive control as 100%.

Inhibitory Activity (%)=(([Radioactivity of Control Group (cpm)]−[Radioactivity of Group with Addition of Subject Sample (cpm)])/([Radioactivity of Control Group (cpm)]−[Radioactivity of Group with Addition of 10 μM FR179254 (cpm)]))×100

The results are shown in Table 2. In other words, Table 2 shows the inhibitory activity for ACAT in each final concentration of each of the subject samples, and the remarkable inhibitory activity for ACAT was found in each of the subject samples described in the table.

TABLE 2

| | Final | Inhibitory |
|---|---|---|
| | Concentration | Activity for |
| Subject Sample | (%) | ACAT (%) |
| Ethanol Extract Fraction from Root of | 0.1 | 105 |
| Peucedanum japonicum | 0.05 | 71 |
| | 0.025 | 48 |
| Ethanol Extract Fraction from Leaf of | 0.1 | 61 |
| Peucedanum japonicum | 0.05 | 66 |
| | 0.025 | 48 |
| | Final | Inhibitory |
| | Concentration | Activity for |
| Subject Sample | (μM) | ACAT (%) |
| 3′-acetoxy-4′-senecioyloxy- | 100 | 87 |
| 3′,4′-dihydroseselin | 50 | 65 |
| | 25 | 40 |

### INDUSTRIAL APPLICABILITY

According to the present invention, there is provided a medicament, food or feed for treating or preventing a disease requiring an anti-foaming action for a cell and/or an inhibitory action for ACAT in treatment or prevention comprising a processed product derived from Peucedanum japonicum, 3′-acetoxy-4′-senecioyloxy-3′,4′-dihydroseselin, a derivative thereof, and/or pharmaceutically acceptable salts thereof. The medicament is useful as a therapeutic agent or prophylactic agent for arteriosclerosis, hyperlipemia, or a disease caused by these diseases as a causal factor. Also, by taking the food as foodstuff on a daily basis, symptoms of the above-mentioned disease can be ameliorated or prevented. Therefore, the food comprising an effective ingredient of the present invention is a functional food useful in maintaining homeostasis of a living body according to an anti-foaming action for a cell and/or an inhibitory action for ACAT.

1. A therapeutic agent or prophylactic agent for a disease requiring an anti-foaming action for a cell and/or an inhibitory action for acyl-CoA cholesterol acyltransferase in treatment or prevention, characterized in that the therapeutic agent or prophylactic agent comprises as an effective ingredient a processed product derived from Peucedanum japonicum.

2. The therapeutic agent or prophylactic agent according to claim 1, wherein the processed product derived from Peucedanum japonicum is a processed product comprising one or more kinds selected from the group consisting of 3′-acetoxy-4′-senecioyloxy-3′,4′-dihydroseselin, a derivative thereof, and pharmaceutically acceptable salts thereof.

3. An anti-foaming agent for a cell, characterized in that the anti-foaming agent comprises as an effective ingredient a processed product derived from Peucedanum japonicum.

4. The anti-foaming agent for a cell according to claim 3, wherein the processed product derived from Peucedanum japonicum is a processed product comprising one or more kinds selected from the group consisting of 3′-acetoxy-4′-senecioyloxy-3′,4′-dihydroseselin, a derivative thereof, and pharmaceutically acceptable salts thereof.

5. An inhibitory agent for acyl-CoA cholesterol acyltransferase, characterized in that the inhibitory agent comprises as an effective ingredient a processed product derived from Peucedanum japonicum.

6. The inhibitory agent for acyl-CoA cholesterol acyltransferase according to claim 5, wherein the processed product derived from Peucedanum japonicum is a processed product comprising one or more kinds selected from the group consisting of 3′-acetoxy-4′-senecioyloxy-3′,4′-dihydroseselin, a derivative thereof, and pharmaceutically acceptable salts thereof.

7. A food or feed for anti-foaming for a cell and/or an inhibition for acyl-CoA cholesterol acyltransferase, characterized in that the food or feed comprises as an effective ingredient a processed product derived from Peucedanum japonicum.

8. The food or feed according to claim 7, wherein the processed product derived from Peucedanum japonicum is a processed product comprising one or more kinds selected from the group consisting of 3′-acetoxy-4′-senecioyloxy-3′,4′-dihydroseselin, a derivative thereof, and pharmaceutically acceptable salts thereof.

9. A therapeutic agent or prophylactic agent for a disease requiring an anti-foaming action for a cell and/or an inhibitory action for acyl-CoA cholesterol acyltransferase in treatment or prevention, characterized in that the therapeutic agent or prophylactic agent comprises as an effective ingredient one or more kinds selected from the group consisting of 3′-acetoxy-4′-senecioyloxy-3′,4′-dihydroseselin, a derivative thereof, and pharmaceutically acceptable salts thereof.

10. An anti-foaming agent for a cell, characterized in that the anti-foaming agent comprises as an effective ingredient one or more kinds selected from the group consisting of 3′-acetoxy-4′-senecioyloxy-3′,4′-dihydroseselin, a derivative thereof, and pharmaceutically acceptable salts thereof.

11. An inhibitory agent for acyl-CoA cholesterol acyltransferase, characterized in that the inhibitory agent comprises as an effective ingredient one or more kinds selected from the group consisting of 3′-acetoxy-4′-senecioyloxy-3′,4′-dihydroseselin, a derivative thereof, and pharmaceutically acceptable salts thereof.

12. A food or feed for anti-foaming for a cell and/or an inhibition for acyl-CoA cholesterol acyltransferase, characterized in that the food or feed comprises as an effective ingredient one or more kinds selected from the group consisting of 3′-acetoxy-4′-senecioyloxy-3′,4′-dihydroseselin, a derivative thereof, and pharmaceutically acceptable salts thereof.

13. A method of treating or preventing a disease requiring an anti-foaming action for a cell and/or an inhibitory action for acyl-CoA cholesterol acyltransferase in treatment or prevention, comprising administering to a subject an effective amount of a processed product derived from Peucedanum japonicum.

14. Use of a processed product derived from Peucedanum japonicum for manufacturing of a therapeutic agent or prophylactic agent for a disease requiring an anti-foaming action for a cell and/or an inhibitory action for acyl-CoA cholesterol acyltransferase in treatment or prevention.

15. A method of treating or preventing a disease requiring an anti-foaming action for a cell and/or an inhibitory action for acyl-CoA cholesterol acyltransferase in treatment or prevention, comprising administering to a subject an effective amount of one or more kinds selected from the group consisting of 3′-acetoxy-4′-senecioyloxy-3′,4′-dihydroseselin, a derivative thereof, and pharmaceutically acceptable salts thereof.

16. Use of one or more kinds selected from the group consisting of 3′-acetoxy-4′-senecioyloxy-3′,4′-dihydroseselin, a derivative thereof, and pharmaceutically acceptable salts thereof for manufacturing of a therapeutic agent or prophylactic agent for a disease requiring an anti-foaming action for a cell and/or an inhibitory action for acyl-CoA cholesterol acyltransferase in treatment or prevention.
